(19) United States
(12) Patent Application Publication
(10) Pub. No.: US 2004/0193130 A1
(43) Pub. Date: Sep. 30, 2004

(54) Disposable absorbent article having fecal collection portion

(76) Inventors: Raoul G. Fima, Oceanside, CA (US); Thomas Gagliano, Huntington Beach, CA (US)

Correspondence Address: william b. noll, 402 anemone street, panama city beach, FL, 32413, US

(21) Appl. No.: 10/402,908

(22) Filed: Mar. 28, 2003

(51) Int. Cl. A61F 13/15; A61F 13/20

(52) U.S. Cl. 604/385.01; 604/385.19

(57) ABSTRACT

This invention is particularly suited to the aging population of baby boomers and patients who, having no control of bowel functions, are literally helpless in that regard and accordingly present a serious problem from the standpoint of cleanliness and nursing care. The structure contemplates a disposable diaper which comprises a topsheet, backsheet, and absorbent core. The topsheet has an aperture which is secured to a flange of flexible material such as paper, plastic, soft foam, or textile, treated with a pressure sensitive adhesive so as to adhere to the buttocks of a patient. The flange is also affixed to a vented container and aligned with the alimentary canal of the patient in order to receive fecal material discharged by the patient.

### FIELD OF INVENTION

The present invention relates to disposable personal care articles which absorb and/or retain bodily excrements including disposal personal care absorbent articles such as diapers, adult incontinence products and the like. More particularly, the invention relates to disposable diapers which have the capacity to retain fecal material and which provides for less messiness when a soiled disposable diaper is removed from the wearer.

### BACKGROUND OF THE INVENTION

Infants, children and adult incontinent individuals wear disposable personal care absorbent articles such as diapers, training pants, and incontinent briefs to receive and contain discharged urine and excrement. Disposable personal care absorbent articles such as diapers, training pants, and incontinent briefs are well known in the art. These disposable personal care absorbent articles can function both to retain the discharged materials and to isolate those materials from the body of the wearer as well as from the garments, clothing and bedding of the wearer. Generally, such disposable personal care absorbent articles collect and retain urine and excrement including fecal material and any other waste matter discharged from the alimentary canal and deposited thereon by the wearer.

Typical disposable diapers include a topsheet which faces towards and contacts the body of the wearer and a liquid impervious backsheet that is positioned opposite the topsheet. Between the topsheet and the backsheet is an absorbent core. A typical absorbent core comprises a combination of fibrous material and discrete particles of absorbent gelling material allowing the topsheet to be drained of liquid that contacts it so that the topsheet may acquire and distribute more liquids. Absorbent core absorbs urine or other liquids and transfers these liquids to a storage area keeping the wearer dry even when the disposable diaper is removed from the wearer.

Most disposable diapers, training pants, and incontinent briefs have been specifically configured for handling urine. Dealing with fecal material is simply more difficult than dealing with urine due to the low viscosity of the fecal material. Additionally, most attempts to handle urine do little, if anything, to improve the handling of the low viscosity fecal material which is the emphasis of the present invention. Feces contained in the diaper can harm the skin of the wearer over time and feces leaking from the diaper almost invariably presents unpleasant, messy clean-ups. Thus, several attempts have been made to add features to diapers such as barriers, pockets, spacers, transverse barriers, apertured topsheets and the like to limit the movement of the fecal material across the topsheet and/or to better confine the fecal matter in the diapery. Some attempts to deal with fecal material include disposable diapers with topsheets that have an opening in them for depositing fecal material. Examples of such structures including a relatively large single aperture in the topsheet are disclosed in U.S. Pat. No. 6,423,044 entitled “Disposal Article Having Bodily Waste Isolation Device” issued to Roe, et al. on Jul. 23, 2002; U.S. Pat. No. 6,458,110 entitled “Disposable Article Having An Expandable Component” issued to Lavon, et al. on Oct. 1, 2002; and U.S. Pat. No. 5,624,422 entitled “Absorbent Article Having An Extendable Split Core” issued to Allen on Apr. 29, 1997.

These attempts to deal with fecal material passing through a single aperture have not adequately addressed the issue of messiness. Among the problems experienced with such disposable diapers are leakage of the bodily excrement, contamination of large portions of the wearer's skin with smeared feces, difficulty in cleaning the bodily excrement from the wearer, odor, frustration by the wearer as well as frustration on behalf of caregivers such as nurses, nursing homes, parents, and the like.

There have been a few attempts to deal with registering (properly aligning) the opening in the topsheet of the disposable diaper with the anal opening of the wearer. Registering the opening with the anus is an attempt to assure that fecal material passes through the opening without contacting or being disbursed onto the topsheet. Attempts to properly register the opening in the disposable diaper with the anus of the user are disclosed in, for example, U.S. Pat. No. 6,132,409 entitled “Personal Care Article With Aperture Aligned For Receiving Fecal Material” issued to Vogt, et al. on Oct. 17, 2000, and U.S. Pat. No. 6,454,750 entitled “Personal Care Article With Aperture Aligned For Receiving Fecal Material” issued to Vogt, et al. on Sep. 24, 2002. Often proper alignment may still not be achieved and any misalignment may allow spreading of the excrement onto the topsheet of the disposable diaper which allows for contamination of the wearer's buttocks. Even with proper alignment, often a gap will exist between the disposable diaper and the anus of the wearer. Such a gap can allow fecal material to exude between the topsheet of the disposable diaper and the body of the wearer. Consequently, as the wearer moves around, the fecal material can migrate both longitudinally and transversely along the topsheet of the disposable diaper. Such spreading allows the wearer's skin to become smeared and contaminated with feces.

There have been yet other attempts to improve the construction of such disposable diapers by focusing on the fecal containment reservoir itself. Many structures have attempted to enlarge the containment reservoir upon the entry of fecal material. Some devices expand when contacted by liquids, for example, U.S. Pat. No. 6,346,097 entitled “Personal Care Product With Expandable BM Containment” issued to Blaney on Feb. 12, 2002. Other disposable diapers maintain the walls of the fecal containment reservoir in a compressed state until the compressive force is released allowing the reservoir to expand to an uncompressed state forming a cavity for the fecal material, for example, U.S. Pat. No. 6,458,110 entitled “Disposable Article Having An Expandable Component” issued to Lavon, et al. on Oct 1, 2002, and U.S. Pat. No. 6,423,045 entitled “Disposable Garment Having An Expandable Component” issued to Wise, et al. on Jul. 23, 2002.

Leakage while utilizing such disposable diapers can occur for a number of reasons including one or more of the two following reasons. First, there may be a gap or misalignment of the aperture with the anus of the wearer. In this event, the gap or misalignment can allow the excrement to flow laterally resulting in leakage and its subsequent messiness during cleanup and discomfort to the wearer.

Secondly, excrement that is discharged from the user's body in a rapid manner or that is jointly discharged with bodily gases requires the fecal collection reservoir to expand in a rapid manner. Most disposable diapers allow for the escape of such gases by providing a gap or misalignment/off-set between the anus and the aperture in the disposable diaper which enables these gases to be vented. However, if the aperture is properly registered and it is also sealed against the perianal area of the wearer, the fecal material will not leak, it will enter into the fecal collection reservoir along with any gases discharged from the alimentary canal. This may cause a flexible fecal collection reservoir to balloon outward and subsequent backpressure may cause a gap or misalignment separating the aperture from the perianal area of the wearer. Without an easy method for rapid evacuation of gases from the fecal collection reservoir, such potential pressure differentials may cause any seal between the fecal collection reservoir and the wearer's anal area to become separated causing leakage. If the bowel movement continues after such a gap or misalignment is created, leakage may result.

Consequently, a need exists for a disposable diaper having a fecal collection reservoir that functions as an extension of the alimentary canal. In such a disposable diaper, the need for sealing around the anal region is significant. Additionally, to function as an extension of the alimentary canal, a fecal collection reservoir must rapidly vent any gaseous discharge received into the fecal collection reservoir. The problem of excrement leakage when using disposable diapers is well known, especially in association with explosive bowel movements related to diarrhea or other abdominal ailments.

Accordingly, it is desirable to provide a disposable personal care absorbent article that reduces the leakage of fecal material while also providing for simplified registering of the aperture for the fecal collection reservoir in order to reduce the amount of fecal material remaining on the skin of the wearer once the disposable personal care absorbent article is removed. This issue is even more acute when it involves geriatric patients, especially those in a nursing home environment.

### SUMMARY OF THE INVENTION

In order to solve the problems of the prior art, the present invention is directed to a disposable personal care absorbent article having a fecal collection portion. The disposable personal care absorbent article comprises a disposable diaper having a liquid permeable topsheet with an aperture located therein. A liquid impervious backsheet is joined to the topsheet and an absorbent core is positioned between the topsheet and the backsheet. A fecal collection portion is also included. The fecal collection portion is accessible through the aperture in the topsheet. The fecal collection portion includes a flange and a container. The container is made of a liquid impervious material that is flexible. The flange has a topside with an opening therethrough. At least the topside of the flange is conformable to the buttocks of the wearer. An adhesive is included on the topside of the flange for sealable attachment to the perianal area of the wearer. The container has at least one venting valve located therein. The container is attached to the flange such that excrement passing through the opening enters into the container of the fecal collection portion. The venting valve allows any gases entering into the container to rapidly escape. This provides for a disposable diaper having a fecal collection portion that functions as an extension of the alimentary canal.

### BRIEF DESCRIPTION OF THE DRAWINGS

While the specification concludes with claims particularly pointing out and distinctly claiming the subject matter which is regarded as the present invention, it is believed that the description will be better understood from the following descriptions which are taken in conjunction with the accompanying drawings in which like designations are used to designate substantially identical elements.

FIG. 1 is a plan view of the article made in accordance with the present invention in a flat out state with portions of the structure being cut away to more clearly show the construction of the article;

FIG. 2 shows a sectional view of FIG. 1 taken along line 2-2;

FIG. 3 is a depiction of FIG. 2 when in use by the wearer;

FIG. 4 shows an alternative embodiment of FIG. 2;

FIG. 4A is a blown up partial view of the bottom side of the flange shown in FIG. 4;

FIG. 4B shows an alternative embodiment of FIG. 4A;

FIG. 5 shows a perspective view of a flange in accordance with the present invention;

FIG. 6 shows a side view of the flange of FIG. 5;

FIG. 7 shows a sectional view of FIG. 6 taken along lines 7-7 of FIG. 6;

FIG. 8 shows a partial sectional view of FIG. 1 taken along lines 8-8 of FIG. 1;

FIG. 9 is a blown up view of the venting valve of the present invention shown in FIG. 8;

FIG. 10 shows a plan view of an embodiment of the present invention of FIG. 9 taken along lines 10-10 of FIG. 9;

FIG. 11 shows an alternative embodiment of the present invention of FIG. 10;

FIG. 12 shows an alternative embodiment of the present invention of FIG. 9;

FIG. 13 shows a plan view of the invention of FIG. 12 taken along lines 13-13 of FIG. 12;

FIG. 14 shows an alternative embodiment of the present invention of FIG. 13; and

FIG. 15 shows a perspective view of an article made in accordance with the present invention with portions of the structure being cut away to more clearly show the construction of the article during use by the wearer.

### DETAILED DESCRIPTION OF THE INVENTION

As used herein, the term “absorbent article” refers to devices which absorb and contain body exudates, and more specifically, refers to devices which are placed against or in proximity to the body of the wearer to absorb and contain the various exudates discharged from the body.

The term “disposable” is used herein to describe absorbent articles which generally are not intended to be laundered or otherwise restored or reused as an absorbent article (i.e., they are intended to be discarded after a single use and, preferably, to be recycled, composted or otherwise disposed of in an environmentally compatible manner).

As used herein, the term “disposed” is used to mean that an element(s) of diaper 20 is formed (joined or positioned) in a particular place or position as a unitary structure with other elements of diaper 20 or as a separate element joined to another element of diaper 20.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

A “unitary” absorbent article refers to absorbent articles which are formed of separate parts united together to form a coordinated entity so that they do not require separate manipulative parts like a separate holder and liner.

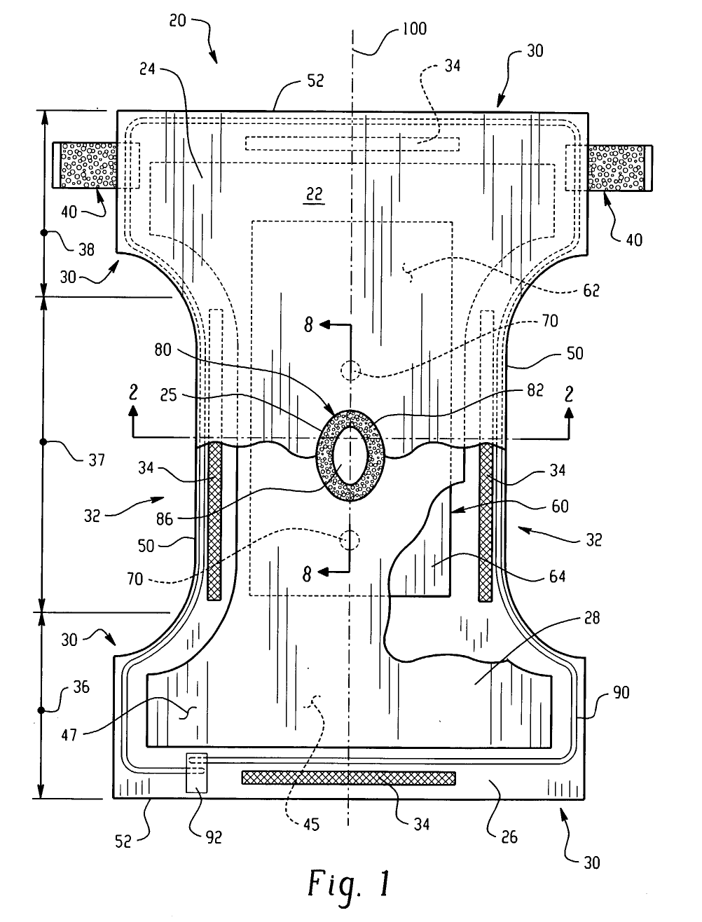

A preferred embodiment of an absorbent article of the present invention is the unitary disposable absorbent article, diaper 20, shown in FIG. 1. (As used herein, the term “diaper” refers to an absorbent article generally worn by infants and incontinent persons about the lower torso.) However, the present invention is also applicable to other absorbent articles such as incontinence briefs, incontinence undergarments, absorbent inserts, diaper 20 holders and liners, feminine hygiene garments, wipes, mops, bandages and the like. The present invention is also applicable to absorbent or nonabsorbent feces collection devices which can be separately applied to the wearer's perianal region.

FIG. 1 is a plan view of diaper 20 of the present invention in a flat-out, state with portions of the structure being cut-away to more clearly show the construction of diaper 20. The portion of diaper 20 which faces the wearer is oriented towards the viewer. As shown in FIG. 1, diaper 20 preferably comprises a liquid permeable topsheet 24; a liquid impervious backsheet 26; an absorbent core 28, which is preferably positioned between at least a portion of topsheet 24 and backsheet 26; side panels 30; leg cuff 32; an elastic member 34; and a fastening system 40 generally designated 40. Diaper 20 is shown in FIG. 1 to have a first or front waist region 36, a second or rear waist region 38 opposed to first waist region 36 and a crotch region 37 located between first waist region 36 and second waist region 38. The periphery of diaper 20 is defined by the outer edges of diaper 20 in which longitudinal edges 50 run generally parallel to longitudinal centerline 100 of diaper 20 and end edges 52 run between longitudinal edges 50 generally perpendicular to longitudinal centerline 100 of diaper 20.

Chassis 22 of diaper 20 comprises the main body of diaper 20. Chassis 22 comprises at least a portion of absorbent core 28 and preferably an outer covering layer including topsheet 24 and backsheet 26. If the absorbent article comprises a separate holder and a liner, chassis 22 generally comprises the holder and the liner. (For example, the holder may comprise one or more layers of material to form the outer cover of the article and the liner may comprise an absorbent assembly including a topsheet 24, a backsheet 26, and an absorbent core 28. In such cases, the holder and/or the liner may include a fastening element which is used to hold the liner in place throughout the time of use.) For unitary absorbent articles, chassis 22 comprises the main structure of diaper 20 with other features added to form the composite diaper 20 structure.

While topsheet 24, backsheet 26, and absorbent core 28 may be assembled in a variety of well known configurations, preferred diaper 20 configurations are described generally in U.S. Pat. No. 3,860,003 entitled “Contractible Side Portions for Disposable Diaper” which issued to Kenneth B. Buell on Jan. 14, 1975; U.S. Pat. No. 5,151,092 issued to Buell on Sep. 9, 1992; and U.S. Pat. No. 5,221,274 issued to Buell on Jun. 22, 1993; and U.S. Pat. No. 5,554,145 entitled “Absorbent Article With Multiple Zone Structural Elastic-Like Film Web Extensible Waist Feature” which issued to Roe et al. on Sep. 10, 1996; U.S. Pat. No. 5,569,234 entitled “Disposable Pull-On Pant” which issued to Buell et al. on Oct. 29, 1996; and U.S. Pat. No. 5,580,411 entitled “Zero Scrap Method For Manufacturing Side Panels For Absorbent Articles” which issued to Nease et al. on Dec. 3, 1996; each of which is incorporated herein by reference.

Backsheet 26 is generally that portion of diaper 20 positioned adjacent garment facing surface 45 of absorbent core 28 which prevents the excrements or urine absorbed and contained therein from soiling articles which may contact diaper 20, such as bed sheets and undergarments. In preferred embodiments, backsheet 26 is impervious to liquids (e.g., urine) and comprises a thin plastic film such as a thermoplastic film having a thickness of about 0.012 mm (0.5 mil) to about 0.051 mm (2.0 mils). Suitable backsheet 26 films include those manufactured by Tredegar Industries Inc. of Terre Haute, Ind. and sold under the trade names X15306, X10962 and X10964. Other suitable backsheet 26 materials may include breathable materials which permit vapors and gases to escape from diaper 20 while still preventing excrements or urine from passing through backsheet 26. Exemplary breathable materials may include materials such as woven webs, non-woven webs, composite materials such as film-coated non-woven webs, and micro porous films such as manufactured by Mitsui Toatsu Co., of Japan under the designation ESPOIR NO and by Exxon Chemical Co., of Bay City, Tex., under the designation EXXAIRE. Suitable breathable composite materials comprising polymer blends are available from Clopay Corporation, Cincinnati, Ohio under the name HYTREL blend P18-3097. Some breathable composite materials are described in greater detail in PCT Application No. WO 95/16746, published on Jun. 22, 1995 in the name of E.I. DuPont and U.S. Pat. No. 5,865,823 issued to Curro on Feb. 2, 1999. Other breathable backsheets including non-woven webs and apertured formed films are described in U.S. Pat. No. 5,571,096 issued to Dobrin et al. on Nov. 5, 1996. Each of these references is hereby incorporated by reference herein.

Backsheet 26, or any portion thereof, may be elastically extensible in one or more directions. In one embodiment, backsheet 26 may comprise a structural elastic-like film (“SELF”) web as described in U.S. Pat. No. 5,518,801 entitled Web Materials Exhibiting Elastic-Like Behavior, which issued to Chappell, et, al. on May 21, 1996, which is incorporated herein by reference. In alternate embodiments, backsheet 26 may comprise elastomeric films, foams, strands, or combinations of these or other suitable materials with non-wovens or synthetic films.

Backsheet 26 maybe joined to topsheet 24, absorbent core 28 or any other element of diaper 20 by any attachment means known in the art. For example, the attachment means may include a uniform continuous layer of adhesive, a patterned layer of adhesive, or an array of separate lines, spirals, or spots of adhesive. Alternatively, the attachment means may comprise heat bonds, pressure bonds, ultrasonic bonds, dynamic mechanical bonds, or any other suitable attachment means or combinations of these attachment means as are known in the art.

Topsheet 24 is preferably compliant, soft feeling, and non-irritating to the wearer's skin. Further, at least a portion of topsheet 24 is liquid permeable, permitting liquids to readily penetrate through its thickness. A suitable topsheet 24 may be manufactured from a wide range of materials, such as porous foams; reticulated foams; apertured plastic films; or woven or non-woven webs of natural fibers (e.g., wood or cotton fibers), synthetic fibers (e.g., polyester or polypropylene fibers), or a combination of natural and synthetic fibers. If topsheet 24 includes fibers, the fibers may be spunbond, carded, wet-laid, meltblown, hydroentangled, or otherwise processed as is known in the art. One suitable topsheet 24 comprising a web of staple length polypropylene fibers is manufactured by Veratec, Inc., a Division of International Paper Company, of Walpole, Mass. under the designation P-8.

Suitable formed film topsheets 24 are described in U.S. Pat. No. 3,929,135, entitled “Absorptive Structures Having Tapered Capillaries”, which issued to Thompson on Dec. 30, 1975; U.S. Pat. No. 4,324,246 entitled “Disposable Absorbent Article Having A Stain Resistant Topsheet”, which issued to Mullane, et al. on Apr. 13, 1982; U.S. Pat. No. 4,342,314 entitled “Resilient Plastic Web Exhibiting Fiber-Like Properties”, which issued to Radel, et al. on Aug. 3, 1982; U.S. Pat. No. 4,463,045 entitled “Macroscopically Expanded Three-Dimensional Plastic Web Exhibiting Non-Glossy Visible Surface and Cloth-Like Tactile Impression”, which issued to Ahr, et al. on Jul. 31, 1984; and U.S. Pat. No. 5,006,394 “Multilayer Polymeric Film” issued to Baird on Apr. 9, 1991. Other suitable topsheets 24 are made in accordance with U.S. Pat. Nos. 4,609,518 and 4,629,643 which issued to Curro et al. on Sep. 2, 1986 and Dec. 16, 1986, respectively, and both of which are incorporated herein by reference. Such formed films are available from The Procter & Gamble Company of Cincinnati, Ohio as “DRI-WEAVE” and from Tredegar Corporation of Terre Haute, Ind. as “CLIFF-T.”

Topsheet 24 may be made of a hydrophobic material or be treated to be hydrophobic in order to isolate the wearer's skin from liquids contained in absorbent core 28. If topsheet 24 is made of a hydrophobic material, preferably at least the upper surface of topsheet 24 is treated to be hydrophilic so that liquids will transfer through topsheet 24 more rapidly. This diminishes the likelihood that body urine will flow off topsheet 24 rather than being drawn through topsheet 24 and being absorbed by absorbent core 28. Topsheet 24 can be rendered hydrophilic by treating it with a surfactant or by incorporating a surfactant into topsheet 24. Suitable methods for treating topsheet 24 with a surfactant or incorporating a surfactant in a topsheet 24 are described in U.S. Pat. No. 4,988,344 entitled “Absorbent Articles with Multiple Layer Absorbent Layers” issued to Reising, et al. on Jan. 29, 1991 and U.S. Pat. No. 4,988,345 entitled “Absorbent Articles with Rapid Acquiring Absorbent Cores” issued to Reising on Jan. 29, 1991, and U.S. Statutory Invention Registration No. H 1670, published on Jul. 1, 1997 in the names of Aziz et al. Each of these references is hereby incorporated by reference herein. Alternatively, topsheet 24 may include an apertured web or film which is hydrophobic. This may be accomplished eliminating the hydrophilizing treatment step from the production process and/or applying a hydrophobic treatment to topsheet 24, such as a polytetraflouroethylene compound like SCOTCHGUARD or a hydrophobic lotion composition, as described below. In such embodiments, it is preferred that the apertures be large enough to allow the penetration of aqueous fluids like urine without significant resistance.

Any portion of topsheet 24 may be coated with a lotion as is known in the art. Examples of suitable lotions include those described in U.S. Pat. Nos. 5,607,760 entitled “Disposable Absorbent Article Having A Lotioned Topsheet Containing an Emollient and a Polyol Polyester Immobilizing Agent” which issued to Roe on Mar. 4, 1997; U.S. Pat. No. 5,609,587 entitled “Diaper Having A Lotion Topsheet Comprising A Liquid Polyol Polyester Emollient And An Immobilizing Agent” which issued to Roe on Mar. 11, 1997; U.S. Pat. No. 5,635,191 entitled “Diaper Having A Lotioned Topsheet Containing A Polysiloxane Emollient” which issued to Roe et al. on Jun. 3, 1997; and U.S. Pat. No. 5,643,588 entitled “Diaper Having A Lotioned Topsheet” which issued to Roe et al. on Jul. 1, 1997. The lotion may function alone or in combination with another agent as the hydrophobizing treatment described above. Topsheet 24 may also include or be treated with antibacterial agents, some examples of which are disclosed in PCT Publication No. WO 95/24173 entitled “Absorbent Articles Containing Antibacterial Agents in the Topsheet For Odor Control” which was published on Sep. 14, 1995 in the name of Theresa Johnson. Further, topsheet 24, backsheet 26 or any portion of topsheet 24 or backsheet 26 may be embossed and/or matte finished to provide a more cloth like appearance.

Topsheet 24 is preferably positioned adjacent body surface 47 of absorbent core 28 and may be joined thereto and/or to backsheet 26 by any attachment means known in the art. Suitable attachment means are described above with respect to means for joining backsheet 26 to other elements of diaper 20.

Absorbent core 28 may comprise any absorbent material known in the art. Absorbent core 28 may be manufactured in a wide variety of sizes and shapes (e.g., rectangular, hourglass, “T”-shaped, asymmetric, etc.) and may comprise a wide variety of liquid-absorbent materials commonly used in disposable diapers and other absorbent articles such as comminuted wood pulp, which is generally referred to as airfelt. Examples of other suitable absorbent materials include creped cellulose wadding; meltblown polymers, including conform; chemically stiffened, modified or cross-linked cellulosic fibers; tissue, including tissue wraps and tissue laminates; absorbent foams; absorbent sponges; super absorbent polymers; absorbent gelling materials; or any other known absorbent material or combinations of materials.

The configuration and construction of absorbent core 28 may also be varied (e.g., absorbent core 28(s) or other absorbent structure(s) may have varying caliper zones, a hydrophilic gradient, a super absorbent gradient, or lower average density and lower average basis weight acquisition zones; or may comprise one or more layers or structures). However, the total absorbent capacity of absorbent core 28 should be compatible with the design loading and the intended use of diaper 20.

Exemplary absorbent structures for use as absorbent core 28 are described in U.S. Pat. No. 4,610,678 entitled “High-Density Absorbent Structures” issued to Weisman et al. on Sep. 9, 1986; U.S. Pat. No. 4,673,402 entitled “Absorbent Articles With Dual-Layered Cores” issued to Weisman et al. on Jun. 16, 1987; U.S. Pat. No. 4,834,735, entitled “High Density Absorbent Members Having Lower Density and Lower Basis Weight Acquisition Zones”, issued to Alemany et al. on May 30, 1989; U.S. Pat. No. 4,888,231 entitled “Absorbent Core Having A Dusting Layer” issued to Angstadt on Dec. 19, 1989; U.S. Pat. No. 5,137,537 entitled “Absorbent Structure Containing Individualized, Polycarboxylic Acid Crosslinked Wood Pulp Cellulose Fibers” which issued to Herron et al. on Aug. 11, 1992; U.S. Pat. No. 5,147,345 entitled “High Efficiency Absorbent Articles For Incontinence Management” issued to Young et al. on Sep. 15, 1992; U.S. Pat. No. 5,342,338 entitled “Disposable Absorbent Article For Low-Viscosity Fecal Material” issued to Roe on Aug. 30, 1994; U.S. Pat. No. 5,260,345 entitled “Absorybent Foam Materials For Aqueous Body Fluids and Absorbent Articles Containing Such Materials” issued to DesMarais et al. on Nov. 9, 1993; U.S. Pat. No. 5,387,207 entitled “Thin-Until-Wet Absorbent Foam Materials For Aqueous Body Fluids And Process For Making Same” issued to Dyer et al. on Feb. 7, 1995; and U.S. Pat. No. 5,625,222 entitled “Absorbent Foam Materials For Aqueous Fluids Made From high Internal Phase Emulsions Having Very High Water-To-Oil Ratios” issued to DesMarais et al. on Jul. 22, 1997. Each of these patents is incorporated herein by reference.

Diaper 20 may also comprise one or more elastic members 34 to help provide improved fit and containment. Elastic member 34 may be constructed in a number of different configurations including those described in U.S. Pat. No. 4,515,595 issued to Kievit et al. on May 7, 1985; U.S. Pat. No. 4,710,189 issued to Lash on Dec. 1, 1987; U.S. Pat. No. 5,151,092 issued to Buell on Sep. 9, 1992; and U.S. Pat. No. 5,221,274 issued to Buell on Jun. 22, 1993. Other suitable waist configurations may include waistcap features such as those described in U.S. Pat. No. 5,026 364 issued to Robertson on Jun. 25, 1991 and U.S. Pat. No. 4,816,025 issued to Foreman on Mar. 28, 1989. All of the above mentioned references are incorporated herein by reference.

Diaper 20 may also include a fastening system 40. Fastening system 40 preferably maintains first waist region 36 and second waist region 38 in an overlapping configuration so as to provide lateral tensions about the circumference of diaper 20 to hold diaper 20 on the wearer. Fastening system 40 preferably comprises tape tabs and/or hook and loop fastening components, although any other known fastening means are generally acceptable. Some exemplary fastening systems are disclosed in U.S. Pat. No. 3,848,594 entitled “Tape Fastening System for Disposable Diaper” issued to Buell on Nov. 19, 1974; U.S. Pat. No. 4,662,875 entitled “Absorbent Article” issued to Hirotsu et al. on May 5, 1987; U.S. Pat. No. 4,846,815 entitled “Disposable Diaper Having An Improved Fastening Device” issued to Scripps on Jul. 11, 1989; U.S. Pat. No. 4,894,060 entitled “Disposable Diaper With Improved Hook Fastener Portion” issued to Nestegard on Jan. 16, 1990; U.S. Pat. No. 4,946,527 entitled “Pressure-Sensitive Adhesive Fastener And Method of Making Same” issued to Battrell on Aug. 7, 1990; and the herein before referenced U.S. Pat. No. 5,151,092 issued to Buell on Sep. 9, 1992; and U.S. Pat. No. 5,221,274 issued to Buell on Jun. 22, 1993. Fastening system 40 may also provide a means for holding the article in a disposal configuration as disclosed in U.S. Pat. No. 4,963,140 issued to Robertson et al. on Oct. 16, 1990. Each of these patents is incorporated herein by reference. Some exemplary hooks are available from Aplix under the trade names 960E and 960D. Exemplary suitable loops are available from 3M under the trade name EBL and from Guilford under the trade designation 18904. In alternative embodiments, opposing sides of the garment may be seamed or welded to form a pant so as to allow the article to be used as a pull-on type diaper 20, such as a training pant.

Diaper 20 may also include side panels 30 constructed and joined to the chassis in any suitable configuration. Examples of diapers with elasticized side panels 30 are disclosed in U.S. Pat. No. 4,857,067, entitled “Disposable Diaper Having Shirred Ears” issued to Wood, et al. on Aug. 15, 1989; U.S. Pat. No. 4,381,781 issued to Sciaraffa, et al. on May 3, 1983; U.S. Pat. No. 4,938,753 issued to Van Gompel, et al. on Jul. 3, 1990; the herein before referenced U.S. Pat. No. 5,151,092 issued to Buell on Sep. 9, 1992; and U.S. Pat. No. 5,221,274 issued to Buell on Jun. 22, 1993; U.S. Pat. No. 5,669,897 issued to LaVon, et al. on Sep. 23, 1997 entitled “Absorbent Articles Providing Sustained Dynamic Fit”; EPO Publication No. WO 95/13775 A1, published May 26, 1995 entitled “Absorbent Article With Multi-Directional Extensible Side Panels”; each of which is incorporated herein by reference.

Diaper 20 preferably further includes leg cuff 32 to help provide improved containment of liquids and other body excrements or urine. Leg cuff 32 may also be referred to as leg bands, side flaps, barrier cuffs, or elastic cuffs. U.S. Pat. No. 3,860,003 describes a disposable diaper 20 which provides a contractible leg opening having a side flap and one or more elastic members 34 to provide an elasticized leg cuff 32 (a gasketing cuff). U.S. Pat. Nos. 4,808,178 and 4,909,803 issued to Aziz et al. on Feb. 28, 1989 and Mar. 20, 1990, respectively, describe disposable diapers having “stand-up” elasticized flaps (barrier cuffs) which improve the containment of the leg regions. U.S. Pat. Nos. 4,695,278 and 4,795,454 issued to Lawson on Sep. 22, 1987 and to Dragoo on Jan. 3, 1989, respectively, describe disposable diapers having dual cuffs, including gasketing cuffs and barrier cuffs. In some embodiments, it may be desirable to treat all or a portion of leg cuff 32 with a lotion, as described above.

The article of the present invention may also include a portion for receiving and containing excrement. Diaper 20 of the present invention is shown in FIG. 1 with portions of the structure being cut-away to more clearly show the construction of diaper 20. Diaper 20 preferably comprises a liquid permeable topsheet 24; a liquid impervious backsheet 26 and an absorbent core 28 which is preferably disposed between at least a portion of topsheet 24 and backsheet 26. A fecal collection portion 60 is disposed between topsheet 24 and backsheet 26 and more preferably is disposed between absorbent core 28 and backsheet 26.

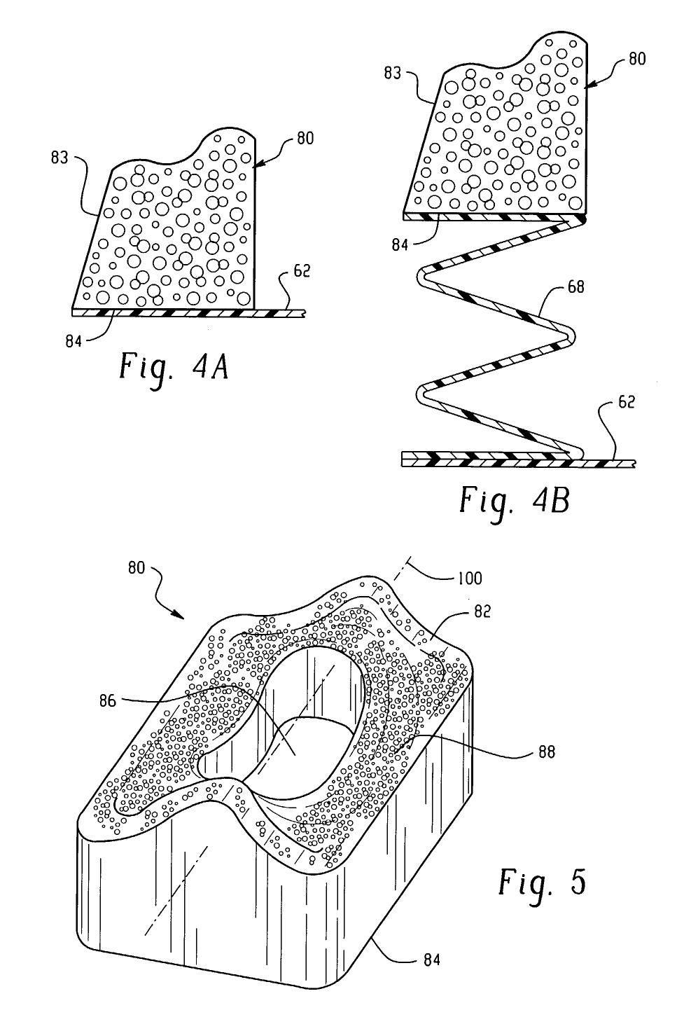

FIG. 1 illustrates a first embodiment of a fecal collection portion 60 according to the present invention, generally designated by reference number 60. Generally, fecal collection portion 60 comprises a thermoplastic layer affixed to backsheet 26. Alternatively, fecal collection portion 60 can be in the form of a bag 63 (shown in FIG. 4) located between topsheet 24 and absorbent core 28, or between absorbent core 28 and backsheet 26. In yet another embodiment fecal collection portion 60 may be in the form of a thermoplastic bag 63 having layers of absorbent core 28 disposed between fecal collection portion 60 and both topsheet 24 and backsheet 26.

Fecal collection portion 60 includes a flange 80 disposed on a container 64. In a preferred embodiment, the container 64 includes at least one sidewall 62 that is liquid impervious. Flange 80 is preferably affixed to a sidewall 62 such that flange 80 extends or protrudes through an aperture 25 in topsheet 24 of diaper 20. Flange 80 has a topside 82 with an opening 86 therethrough. Opening 86 may be substantially oval, round, circular, rectangular or many other shapes allowing fecal material to pass through such an opening 86. In one embodiment of the invention opening 86 has a dimension in the longitudinal direction of about 5.7 centimeters (2.25 inches) and in a transverse dimension of about 3.8 centimeters (1.5 inches). In a preferred embodiment, opening 86 in flange 80 is substantially concentric with aperture 25 in topsheet 24 of diaper 20.

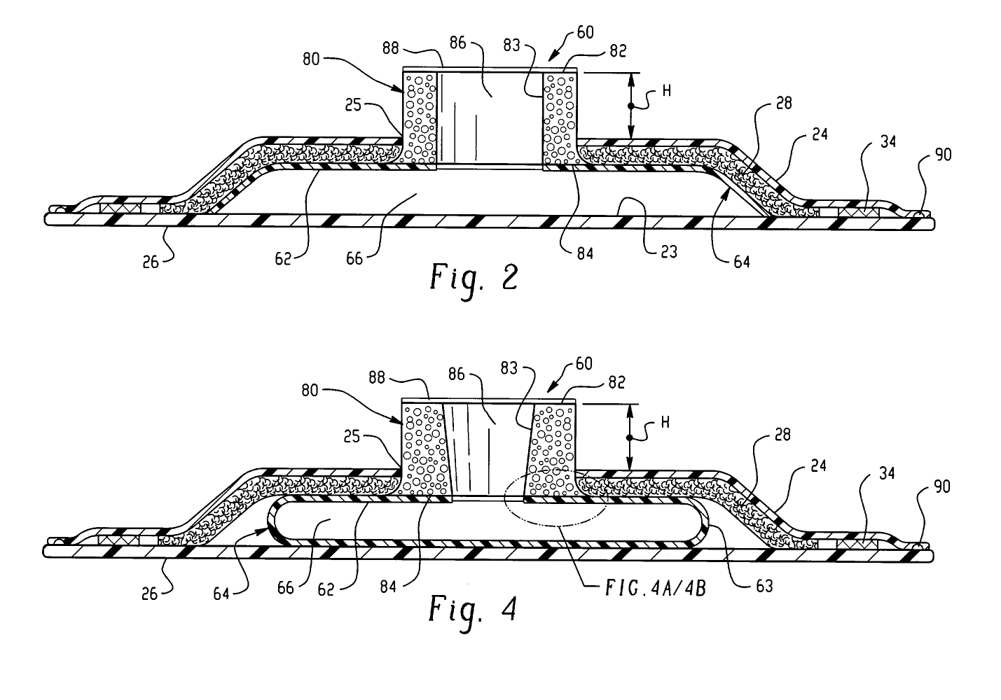

A cross section of diaper 20 is shown in FIG. 2 wherein an internal cavity 66 of the container 64 is shown. Flange 80 of fecal collection portion 60 is shown extending from the container 64 through aperture 25 in topsheet 24 and extends therefrom a given height designated as H. Flange 80 has a topside 82 and a bottom side 84. The topside 82 includes an adhesive layer 88. Adhesive layer 88 is preferably a pressure sensitive type adhesive such as a hypoallergenic, pressure sensitive acrylate adhesive or other adhesive commonly used to releasably adhere or bond items directly in contact with the human skin. Alternatively, adhesive layer 88 may include a protective covering (not shown) in order to avoid the adhesive adhering to undesired areas until diaper 20 is ready for use. Such an adhesive layer 88 can be a two-way tape and alternatively can also be placed on the bottom side 84 of flange 80 in order to affix flange 80 onto sidewall 62 of the container 64.

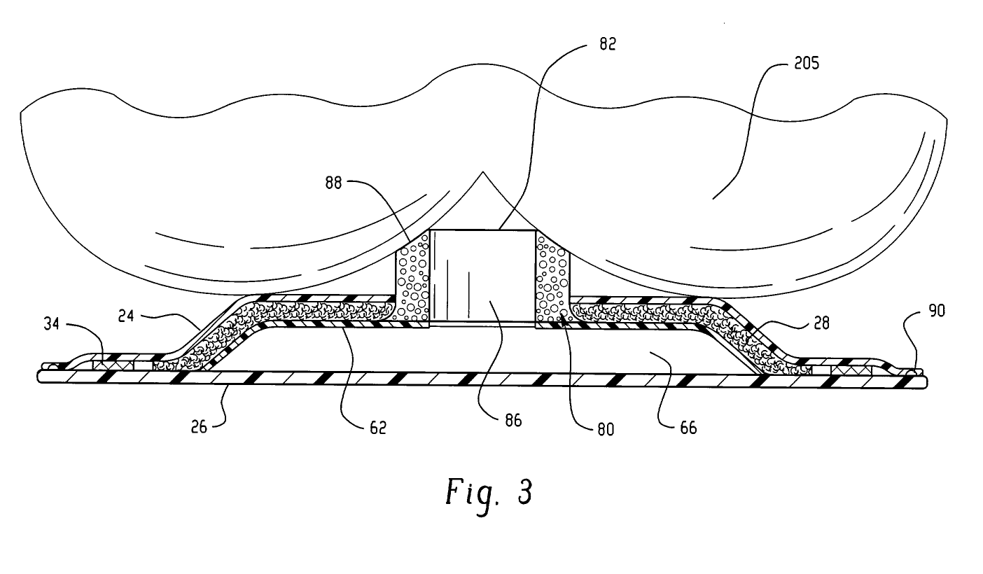

Flange 80 or at least an upper portion thereof is constructed from a conformable or flexible molded foam type material. Such a flexible molded foam is available from the Bayer Corporation of Pittsburgh, Pa. under the name, “Bayfit 582.” Bayfit 582 is a polyurethane system of molded foam medical products that are conformable to the shape of articles which come into contact with it. Such a construction enables flange 80 or at least an upper portion thereof, including at least a portion of height H, to be conformable when pressure is applied thereto. FIG. 3 depicts the application of pressure on flange 80 by buttocks 205 of the wearer of diaper 20. As buttocks 205 come to rest on topsheet 24 of diaper 20, the protective covering of adhesive layer 88 would be removed so the topside 82 of flange 80 can adhere to the perianal area surrounding the anus of the wearer.

In particular, the user will remove any protective covering from adhesive layer 88 and then align opening 86 in flange 80 with the anus of the wearer. As the anus is aligned with opening 86 in flange 80, flange 80 is allowed to conform to buttocks 205 including the gluteal fold or gap allowing flange 80 to come into very close proximity with the anus of the wearer. Adhesive layer 88 adheres to the perianal of the wearer and flange 80 conforms to the contour of the gluteal gap around the anus and therefore maintains opening 86 in such very close proximity with the anus forming an extension of the alimentary canal of the wearer.

In use, any excrement or gases exuding from the anus of the wearer will immediately enter opening 86 in flange 80 and pass therethrough into internal cavity 66 within the container 64 of fecal collection portion 60. Since flange 80 conforms to the perianal area of the wearer and is constructed from a soft foam material, the wearer does not experience any discomfort. The alignment of opening 86 with the anus from the alimentary canal is maintained by adhesive layer 88 on the topside 82 of flange 80. Height H is determined to substantially extend a height sufficient to allow flange 80 to conform with the gluteal gap and Height H is designed to be compatible with the variations in sizes of buttocks 205 and the intended use of wearers of such diapers. In one embodiment of the invention height H can be greater than about 2.5 centimeters (1 inch) in an uncompressed state. Preferably, height H can be greater than about 3.1 centimeters (1.25 inches) and less than about 3.8 centimeters (1.5 inches) in an uncompressed state.

Returning now to FIG. 2, the container 64 is liquid impervious. In particular, fecal collection portion 60 is constructed of a container 64 made from a thermal plastic material that is liquid impervious. The container 64 can be constructed as depicted in FIG. 2 wherein the container 64 is formed by a flexible sidewall 62 joined onto interior surface 23 of backsheet 26. The container 64 and sidewall 62 may be joined to backsheet 26 by any attachment means known in the art. Such attachment means were described above regarding the joining of backsheet 26 to topsheet 24. The joining of sidewall 62 to backsheet 26 may be done in any similar manner. In one embodiment the container 64 and sidewall 62 are constructed from a flexible film of polyurethane that is impervious to liquids. Such a thermal plastic material is available from Stevens Urethane of Easthampton, Mass. under the name Stephens Polyurethane ST-2546-80 and is a polyether aromatic polyurethane type polymer. A container 64 constructed of such a film type material can be maintained in a substantially flat state, that is a state in which the volume of internal cavity 66 is minimized or even non-existent until internal cavity 66 of container 64 is filled with fecal material.

An alternative embodiment to fecal collection portion 60 depicted in FIG. 2 can be seen in FIG. 4. Fecal collection portion 60 includes a container 64 in the form of an enclosed bag 63. Such a bag 63 can be formed from a thermal plastic material and includes a top sidewall 62 which is joined to flange 80 which protrudes and extends through aperture 25 in topsheet 24. In this particular embodiment, fecal collection portion 60 can be formed in a manner that bag 63 is not joined with backsheet 26 and is a wholly separate component positioned between backsheet 26 and absorbent core 28.

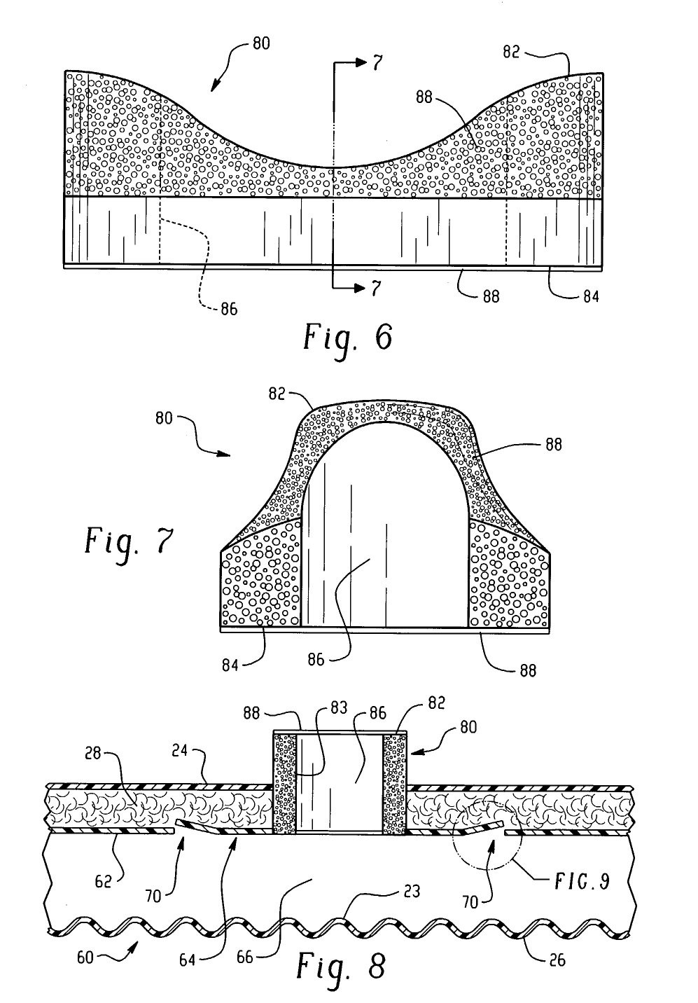

FIG. 5 depicts an embodiment of flange 80 of the present invention. Flange 80 includes a topside 82 and a bottom side 84 with an opening 86 passing therethrough. In this particular embodiment, flange 80 is shown in a substantially rectangular configuration. Opening 86 is generally oval wherein the maximum diameter is parallel with the longitudinal axis of diaper 20. In this embodiment, the topside 82 is contoured to match with the perianal region of buttocks 205 of the wearer. In such a configuration, the user can easily align opening 86 with the anus of the wearer thereby forming an extension of the wearer's alimentary canal. As shown in FIG. 4 the inner annular surface 83 of flange 80 can be substantially cylindrical or conical in contour and preferably is a conical frustum. This contour provides for a wider opening 86 near the topside 82 than at the bottom side 84 of flange 80. Alternatively, the wider opening 86 could be located near the bottom side 84 of flange 80 with opening 86 near the topside 82 being smaller. The bottom side 84 of flange 80 is joined to the container 64 and access into internal cavity 66 is gained through opening 86 in flange 80. FIG. 6 is a side view of flange 80 depicting a somewhat saddle shaped contour of the topside 82. FIG. 7 is a cross section of flange 80 to better enable the reader to understand the conformable nature of flange 80 when placed in the perianal region of the wearer. The material from which flange 80 is constructed allows conforming to contours of the body which enables opening 86 to be placed immediately adjacent the anus of the wearer. Such placement enables adhesive layer 88 to adhere and maintain opening 86 in alignment with the anus of the wearer. Since flange 80 is conformable, the wearer is not discomforted during normal bodily movements.

Adhesive layer 88 on the bottom side 84 of flange 80 enables fecal collection portion 60 to be configured in a manner such that flange 80 can be adhered directly to sidewall 62 of the container 64 as shown in FIG. 4A. Flange 80 can be joined to sidewall 62 of the container 64 using a variety of attachment means known in the art. For example, the attachment means may comprise heat bonds, presssure bonds, ultrasonic bonds, dynamic mechanical bonds, or any other suitable attachment means. Alternatively, as shown in FIG. 4B, flange 80 can be directly affixed to a collar 68 that performs the function of a spacer, expansion joint, or shock absorber. Preferably, the bottom side 84 of flange 80 is joined to sidewall 62 of the container 64 by a flexible annular collar 68. Collar 68 can be easily fabricated in the form of a bellows using the same material as sidewall 62 of the container 64 or some other flexible material that is compatible with flange 80 and sidewall 62. Such a flexible annular collar 68 can also be made as an integral portion of sidewall 62 of the container 64 or collar 68 can be joined to sidewall 62. In this manner flange 80 conforms to the contour of the wearer's buttocks 205 including the gluteal gap region of the perianal area of the wearer surrounding the anus. Additionally, lateral and transverse movements of the wearer are absorbed through the flexibility in collar 68. This flange 80 thereby forms a substantially hermetic seal with the perianal area of the wearer. The flexibility of flange 80, collar 68, or sidewall 62 assures that a substantially hermetic seal is maintained during use by the wearer and consequently, there is no leakage of excrement or gases that exude from the alimentary canal of the wearer. Such a hermetic seal is preferably greater than about 95% effective.

As shown in FIG. 1, the container 64 has at least one venting valve 70 in a sidewall 62. Preferably venting valve 70 is a one-way exhaust valve that is constructed from a flexible material. In one embodiment venting valve 70 is a semicircular slit cut in sidewall 62 of the container 64. In another embodiment venting valve 70 includes a gas permeable membrane 72 and can further include a liquid impervious membrane. Such a gas permeable membrane 72 is available from 3M from the 3M Center of St. Paul, Minn. under the name 3M Single Coated Polyurethane Medical Tape, product number 9842, or Polyurethane Medical Tape, product number 9841. Preferably venting valve 70 is located adjacent to flange 80. In a preferred embodiment venting valve 70 is positioned on longitudinal centerline 100 of diaper 20. Alternatively multiple venting valves 70 can be utilized on opposing sides of flange 80. In FIG. 1 two venting valves 70 are shown aligned on longitudinal centerline 100. While only two venting valves 70 are shown, there can be numerous venting valves 70 positioned in various locations on the container 64 to allow the container 64 to vent internal cavity 66 in a rapid manner. Preferably internal cavity 66 can be instantaneously vented such that any pressure build up in the container 64 is in the range of less than about 1 pound per square inch. Thus it is understood that the container 64 is vented in a rapid or instantaneous manner.

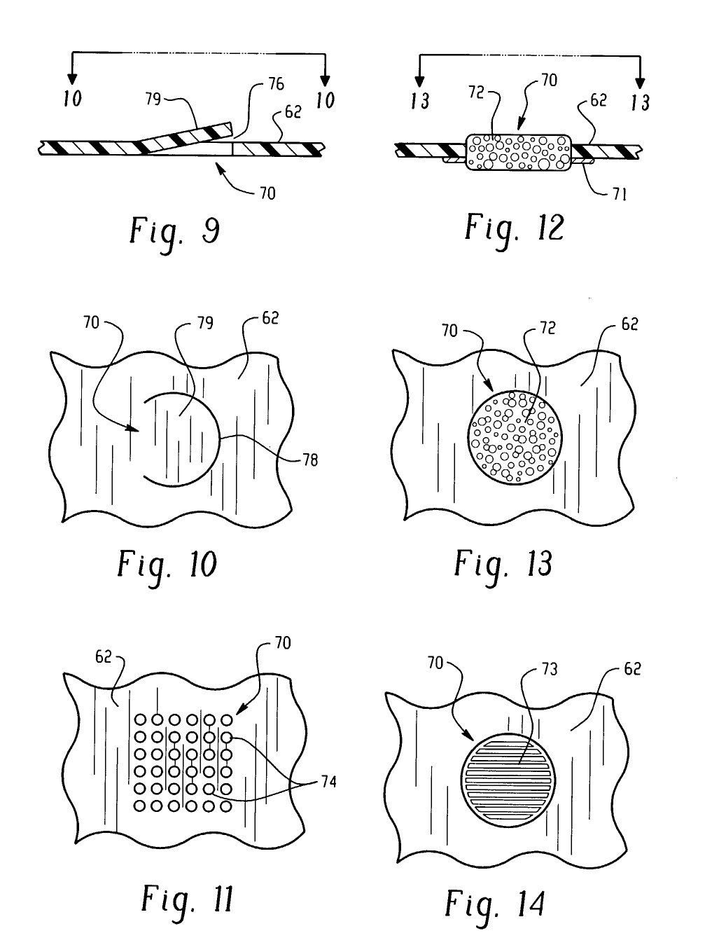

Referring now to FIG. 8 which is a partial cross sectional view taken from FIG. 1. FIG. 8 depicts fecal collection portion 60 including venting valve 70. As shown, backsheet 26 has an interior surface 23 and in a preferred embodiment backsheet 26 is corrugated or rippled allowing for easy expansion of diaper 20 as the wearer moves and bends. During bowel movements or when gases are exuded from the anus of the wearer, such gases pass through opening 86 of flange 80 into internal cavity 66 of the container 64. Any gases that are associated with the excrement passing through opening 86 can escape from internal cavity 66 of the container 64 through venting valve 70. Venting valve 70 is positioned immediately adjacent to flange 80 in order for any pressure to be released without any back flow through opening 86. As depicted in FIG. 8, venting valve 70 is shown as a flapper type valve which is better seen in FIG. 9. As shown in FIG. 9, sidewall 62 of the container 64 includes a vent passage 76 formed by venting valve 70. This vent passage 76 is the effective area formed by a slit or cut 78 through sidewall 62 of the container 64 as gases pass therethrough. In FIG. 10, slit or cut 78 is semi-circular in configuration. Such a semi-circular arrangement allows venting valve 70 to easily open and exhaust any gases entering into the container 64. Alternatively, slit or cut 78 can be in a substantially rectangular or triangular shape, contour and configuration. Additionally, venting valve 70 can be in the form of small holes 74 as shown in FIG. 11. A multiplicity of small holes 74 through sidewall 62 of the container 64 can provide a substantially similar vent passage 76 although this configuration may allow leakage into or out of internal cavity 66 of the container 64. Small holes 74 can be arranged in an array and located in numerous areas or regions of sidewall 62 of the container 64.

Alternatively, venting valve 70 can be in the form of a gas permeable membrane 72 joined to sidewall 62 of the container 64. As depicted in FIG. 12, a gas permeable membrane 72 is attached to sidewall 62 by a mounting member 71. Mounting member 71 may be any form suitable to affix gas permeable membrane 72 to sidewall 62 including, for example, an adhesive, glue, stitching, tape, or other mechanical bonding means or mechanisms. FIG. 13 depicts venting valve 70 having a gas permeable membrane 72 being substantially circular in configuration within sidewall 62 of the container 64. The shape of venting valve 70 can vary from that shown and can be any shape providing a vent passage 76 from internal cavity 66 of the container 64. An alternative venting valve 70 arrangement including mechanical louvers 73 is shown in FIG. 14. Such a venting valve 70 acts as a one-way exhaust valve. Venting valve 70 preferably is constructed of a flexible material that is pliable in order to avoid any inadvertent cutting of sidewall 62 of the container 64 causing a leak into or out of fecal collection portion 60. In a preferred embodiment of venting valve 70 there is a sealing flap 79 (as shown in FIG. 10) that covers vent passage 76 until gases need to escape from internal cavity 66. Such a sealing flap 79 opens to allow gases to escape. In a most preferable arrangement of venting valve 70, venting valve 70 is located immediately adjacent to aperture 25 in topsheet 24 which allows its positioning to be adjacent to flange 80. As depicted in FIG. 1, venting valve 70 is positioned along a longitudinal axis 100 which is the preferable positioning to allow venting valve 70 to align with the gluteal gap of the wearer's buttocks 205 in order to avoid obstruction of vent passage 76. Such a configuration of venting valve 70 allows gases to instantaneously escape from internal cavity 66 of the container 64 of fecal collection portion 60.

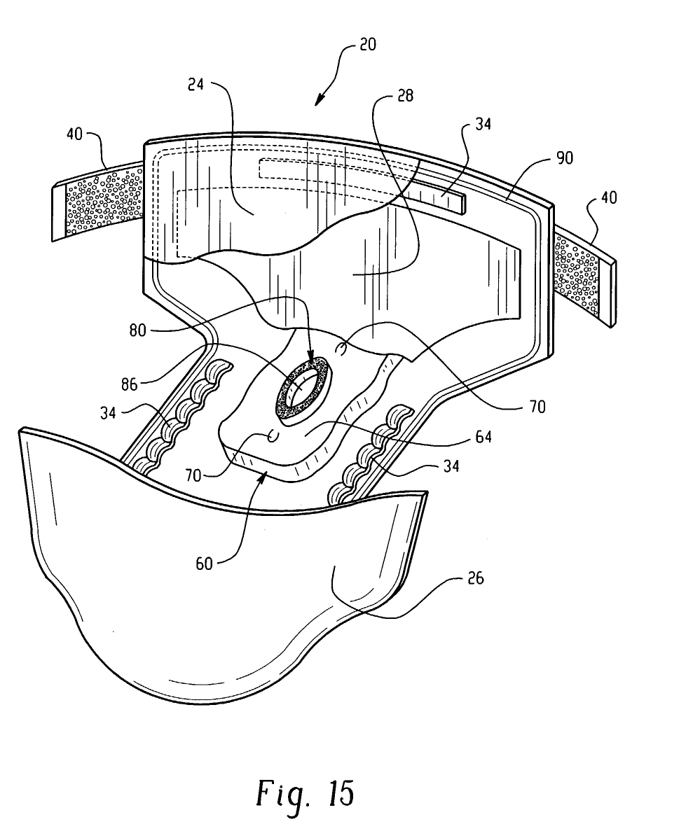

FIG. 15 depicts the disposable diaper 20 in a perspective view depicting the shape of diaper 20 during use with a partial portion of the interior of diaper 20 shown. Since topsheet 24 is removed and a portion of absorbent core 28 is broken away, FIG. 15 shows fecal collection portion 60 located in crotch region 37 of diaper 20. In this view, tear strip 90 is evident around the perimeter of diaper 20. In FIG. 1 pull-tab 92 for tear strip 90 is located in the first waste region 36 of diaper 20. Tear strip 90 is disposed in a manner that when the user grips pull-tab 92 tear strip 90 can easily separate at least a portion of topsheet 24 exposing fecal collection portion 60. This tear strip 90 can be in the form of a weakened section, perforated section, zip lock closure, string, or cord or any other commonly known tear strip 90 for frangible separation of constituent portions of a package.

Having such a tear strip 90 enables the fecal collection portion 60 to be easily separated from the remaining parts of the chassis 22 of disposable diaper 20 and as such the fecal collection portion 60 and its contents can be removed or further examined. This is helpful when the wearer is a patient and the wearer's doctors have a desire to examine the fecal material contained within fecal collection portion 60. Additionally, separating fecal collection portion 60 allows easy disposal or ease of recycling of the various constituent portions of diaper 20. Opening 86 in the topside 82 of flange 80 can be closed by pressing the faces of the topside 82 against one another causing adhesive layer 88 to bond against each other thereby closing opening 86 assuring containment of the fecal material within internal cavity 66.

While particular embodiments and examples and/or individual features of the present invention have been described, it would be obvious to those skilled in the art that various other changes and modifications can be made without departing from the spirit and the scope of this invention. Further, it should be apparent that all combinations of such embodiments and features are possible and can result in preferred executions of the invention. Therefore, the appended claims are intended to cover all such changes and modifications that are within the scope of this invention. Also, although the present invention is described primarily with respect to a disposable diaper 20, the present invention is not limited to this embodiment. The present invention may also be used for example in articles that are applied directly to a wearer prior to the application of a disposable diaper 20 or in place of a disposable diaper 20 in a pull-on diaper 20, a diaper insert, etc. It is therefore intended to cover in the appended claims all such changes and modifications that are within the scope of this invention.

What is claimed is:

1. A disposable personal care absorbent article comprising:
a topsheet, the topsheet having an aperture located therein;
a backsheet, the backsheet joined to the topsheet;
an absorbent core, the absorbent core disposed between the topsheet and the backsheet; and
a fecal collection portion, the fecal collection portion accessible through the aperture in the topsheet, the fecal collection portion including
a flange having a topside with an opening therethrough, the topside having an adhesive layer thereon; and
a container made of a liquid impervious material, the container having at least one venting valve, the container joined to the flange such that any excrement passing through the opening enters into the container and associated gases can escape from the container through the venting valve.

2. The disposable diaper of claim 1 wherein, the flange is a flexible foam material.

3. The disposable diaper of claim 2 wherein, at least the topside of the flange conforms to the contour of the wearer's buttocks including the gluteal gap region.

4. The disposable diaper of claim 1 wherein, the adhesive is a two-way tape.

5. The disposable diaper of claim 1 wherein, the adhesive is a pressure sensitive adhesive.

6. The disposable diaper of claim 3 wherein, the flange forms a substantially hermetic seal with the perianal area of the wearer.

7. The disposable diaper of claim 6 wherein, the substantially hermetic seal is greater than about 95% effective.

8. The disposable diaper of claim 1 wherein, the venting valve is a one-way exhaust valve.

9. The disposable diaper of claim 1 wherein, the venting valve is constructed of a flexible material.

10. The disposable diaper of claim 1 wherein, the venting valve includes a sealing flap that opens to allow gases to escape.

11. The disposable diaper of claim 1 wherein, the venting valve is located adjacent to the flange and is aligned with the longitudinal axis.

12. The disposable diaper of claim 1 wherein, the venting valve includes a gas permeable membrane.

13. The disposable diaper of claim 12 wherein, the gas permeable membrane is liquid impervious.

14. The disposable diaper of claim 1 wherein, the venting valve allows gases to instantaneously escape from the fecal collection portion.

15. The disposable diaper of claim 1 wherein, the venting valve is formed by a multiplicity of small holes in the container.

16. The disposable diaper of claim 1 further comprising a tear strip and a pull-tab that allows easy separation of the topsheet thereby exposing the fecal collection portion.

17. The disposable diaper of claim 1 wherein, the fecal collection portion is easily separable from the remaining components of the disposable diaper.

18. The disposable diaper of claim 17 wherein, the fecal collection portion is affixed to the disposable diaper through a perforated region.

19. The disposable diaper of claim 1 wherein, the container is made of a flexible material.

20. (not entered)

21. The disposable diaper of claim 1 wherein, the flange includes a comfortable upper portion.

22. The disposable diaper of claim 1 wherein, the flange includes an inner annular surface that is substantially conical.

23. The disposable diaper of claim 1 wherein, the flange is attached to the container with a flexible annular collar.

24. The disposable diaper of claim 23 wherein, the flexible annular collar is in the form of a bellows.

25. The disposable diaper of claim 1 wherein, the opening in the flange is substantially concentric with the aperture in the topsheet and the opening is oval.

26. A disposable personal care absorbent article comprising:
a liquid permeable topsheet, the topsheet having an aperture located therein;
a liquid impervious backsheet, the backsheet joined to the topsheet;
an absorbent core, the absorbent core disposed between the topsheet and the backsheet; and
a fecal collection portion, the fecal collection portion accessible through the aperture in the topsheet, the fecal collection portion including
a flange having an upper portion constructed of a conformable molded foam and a topside with an opening therethrough, the topside having an adhesive layer thereon; and
a container made of a liquid impervious material, the container having at least one venting valve, the container joined to the flange such that any excrement passing through the opening enters into the container and associated gases can escape from the container through the venting valve.
